(19) United States
(12) Patent Application Publication
(10) Pub. No.: US 2026/0325369 A1
(43) Pub. Date: Oct. 22, 2026

(54) PACKAGED DISPOSABLE ABSORBENT PRODUCTS

(71) Applicant: THE PROCTER & GAMBLE COMPANY, Cincinnati, OH (US)

(72) Inventors: Nery Vanesa BRESLIN, Hamilton, OH (US); Naomi Ruth NELSON, Cincinnati, OH (US); Sidney Sapphire TRASSER, Florence, KY (US); Curran Riley PUTZ, Wyoming, OH (US)

(21) Appl. No.: 19/222,204

(22) Filed: May 29, 2025

Related U.S. Application Data
(63) Continuation-in-part of application No. 29/999,412, filed on Apr. 16, 2025.
(63) Continuation-in-part of application No. 29/999,404, filed on Apr. 16, 2025.

(51) Int. Cl. A61F 13/551; A61F 13/26

(52) U.S. Cl. CPC A61F 13/55175; A61F 13/266

(57) ABSTRACT

An array of packaged disposable absorbent products including a first package having an education format and a second package having a discretion format, wherein products contained within the first package and the second package include like components. A new user can purchase and use the first package to gain confidence of successful use and thereafter purchase the second package to gain a discretion benefit.

### CROSS REFERENCE TO RELATED APPLICATIONS

This application claims the benefit under 35 U.S.C. § 120, to U.S. Application Ser. No. 29/999,404 filed Apr. 16, 2025, and Ser. No. 29/999,412 filed Apr. 16, 2025, the entire disclosures of which are fully incorporated by reference herein.

### FIELD

The present disclosure relates to packages of disposable absorbent products, arrays of packaged disposable absorbent products, and methods of merchandising disposable absorbent products.

### BACKGROUND

Feminine hygiene products are typically packaged with discretion in mind. Manufacturers have gone to great lengths to include individual products within a wrapper material that includes decorative elements on the outer surface so that consumers can discretely carry feminine hygiene products outside of the home, particularly where the products may be carried in clothing pockets, backpacks, or purses, for example.

Some feminine hygiene products may be confusing, difficult or awkward to properly use by new users. By way of example, applicator tampons require operational aspects that are not necessarily self-explanatory for a new user. And while cartons and bags containing individual products incorporate educational information, once a new user removes individually-wrapped products from their outer packaging for carrying outside of the home, the discrete wrapper material as discussed above fails to inform a new user on attributes, features or aspects of the product.

Accordingly, there is a need for improved packaged disposable absorbent products.

### SUMMARY

Packages of disposable absorbent products are provided. One exemplary package comprises (a) a primary package comprising an applicator tampon; (b) a secondary package comprising a plurality of the primary packages; (c) a design zone on an outer surface of each of the primary packages, the design zone comprising a decorative element; and (d) a product zone on the outer surface of each of the primary packages, the product zone comprising a feature, attribute, and/or operational aspect of the applicator tampon other than size or absorbency level.

Arrays of packaged disposable absorbent articles are also provided. One exemplary array comprises (a) a first package comprising a first primary package containing a first applicator tampon, and a first secondary package comprising a plurality of the first primary packages; and (b) a second package comprising a second primary package containing a second applicator tampon, and a second secondary package comprising a plurality of the second primary packages. An outer surface of each of the first primary packages comprises a color and/or character related to an absorbency level or size of the first applicator tampon; and an aspect related to at least one of an applicator insertion member, an applicator gripping member, and a tampon expulsion member. An outer surface of each of the second primary packages comprises the same color and/or character associated with the first primary packages, but is devoid of the aspect related to at least one of an applicator insertion member, an applicator gripping member, and a tampon expulsion member associated with the first primary packages. The first applicator tampon is the same as the second applicator tampon. And the first secondary package and the second secondary package share a common product name or brand name.

Lastly, methods of merchandising disposable absorbent products are provided. One exemplary method comprises the steps of (a) providing a first package of disposable absorbent products, the first package comprising first primary packages each of which containing a first applicator and a first tampon disposed within the first applicator, and a first secondary package comprising a plurality of the first primary packages; (b) providing a second package of disposable absorbent products, the second package comprising second primary packages each of which containing a second applicator and a second tampon disposed within the second applicator, and a second secondary package comprising a plurality of the second primary packages; and (c) carrying out at least one of (i) offering for sale at the same time the first package of disposable absorbent products and the second package of disposable absorbent products; and (ii) shelving at the same time the first package of disposable absorbent products and the second package of disposable absorbent products. The first tampon is the same as the second a tampon. The first primary packages are configured so that one can see the first applicator contained therein without opening the first primary packages. And the second primary packages are configured so that one cannot see the second applicator contained therein without opening the second primary packages.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a side view of an applicator tampon;

FIG. 2 is a side view of a tampon comprising a primary absorbent and a secondary absorbent;

FIG. 3 is a side view of primary package in the form of a wrapper;

FIG. 4 is perspective view of a secondary package in the form of a carton;

FIG. 5A are secondary packages associated with an array of disposable absorbent products; and

FIG. 5B are primary packages from the secondary packages shown in FIG. 5A.

### DETAILED DESCRIPTION

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent. “Absorbent product” refers to devices that are capable of absorbing and containing a liquid, and more specifically, refers to products that are placed against, in proximity to, or within portions of the body of the wearer to absorb and to contain various exudates discharged from the body. “Array” means a collection of two or more packages that are configured to address a common consumer need, such as absorbing bodily fluid (e.g., menses), and often in a retail setting are displayed in the same aisle such as on the same side of an aisle or generally across an aisle from each other or when offered on-line are offered on the same website. The packages may have the same brand and/or sub-brand (or at least common sub-brand portions) and/or the same trademark registration and/or may have been manufactured by or for a common company. The packages may be available at a common point of sale. More broadly speaking, part of an array may be located in a physical store, while another part of the array is offered on-line. An array is marketed as a line-up of products that may have like packaging elements (e.g., packaging material type, film, paper, dominant color, design theme, same color pallet, design architecture, etc.) and that may convey to consumers that the different individual packages are part of a larger line-up. Arrays often have the same brand name, for example, “Tampax,” and same sub-brand (or portion of the sub-brand), for example, a plurality of packages may have “Pearl,” or a plurality of packages may have “Radiant.” A different product in the array may have the same brand “Tampax” and the sub-brand, or portion of the sub-brand name (these may also be referred to as identifiers or additional information indicia), may be different: a first package may display “Tampax” and may also display “Regular,” and a second package may display “Tampax” and may also display “Super.”

“Brand name” as used herein means a single source identifier, in other words, a brand name identifies a product and/or service as exclusively coming from a single commercial source (that is, company). An example of a brand name is Tampax.

“Disposable” refers to products which are designed to be discarded after a limited use rather than being laundered or otherwise restored for reuse.

“Indicium” and “Indicia” as used herein mean an identifier and/or indicator and/or hint and/or suggestion, of a feature, attribute, or operational aspect, for example, of a disposable absorbent product.

“Non-textual indicia” means a non-text indicia that communicates to a consumer through a consumer's senses. In one example, a non-textual indicia may communicate, even intuitively communicate, to a consumer through sight (visual indicia), through touch (texture indicia), sound (audio indicia) and/or through smell (scent indicia). Nonlimiting examples of non-textual indicia include images, colors, textures, patterns, such as emboss patterns and/or emboss pattern images or images of patterns.

“Textual indicia” means a text indicia, such as a word and/or phrase that communicates to a consumer a property about the disposable absorbent product it is associated with.

The present disclosure provides packages of disposable absorbent products, arrays of packaged disposable absorbent products, and methods of merchandising the same. Disposable absorbent products include, but are not limited to, products worn for feminine hygiene purposes. The products can be designed and configured for capturing and/or absorbing menstrual fluid, urine, anal discharge, bodily fluids, and/or semen. Tampons are one type of feminine hygiene products covered by aspects of the invention.

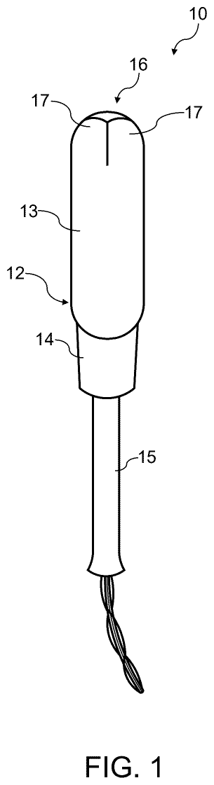

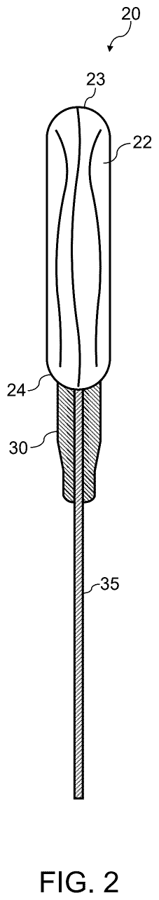

An exemplary applicator tampon 10 is shown in FIG. 1. Applicator tampon 10 includes an applicator 12 and a tampon 20 (shown in FIG. 2) disposed within applicator 12. The exemplary applicator is illustrated with an insertion member 13 (sometimes referred to as a barrel), a gripping member 14, and a tampon expulsion member 15 (sometimes referred to as a plunger or pusher). Insertion member 13 comprises a rounded insertion end 16 that contains a plurality of petals 17 that are enclosed around the tampon when insertion member 13 is inserted into the body, and that flex outwardly thereafter to allow the tampon to be expelled or pushed out by expulsion member 15.

While not shown in FIG. 1, gripping member 14 can include grip enhancing features, such as raised portions, indentations, apertures, and/or high-friction materials such as thermoplastic elastomers.

The insertion member and gripping member can be made as a single component, or as multiple components and then assembled. All three of the applicator members may initially be manufactured as a single component, with the expulsion member separated from the insertion member and gripping member prior to final assembly of the applicator tampon. Exemplary applicator 12 is shown as a full-length applicator that is ready for use when a user removes it from its package. Alternative applicators are designed as compact applicators, wherein a portion of the expulsion member is positioned in a collapsed or shortened state, requiring a user to first extend the expulsion member before it is able to push a tampon out of the insertion member. U.S. Pat. No. 10,383,777 shows an example of a compact applicator.

Insertion member 13, gripping member 14, and tampon expulsion member 15 can be made from various materials and by multiple manufacturing approaches. By way of example only, the applicator members can be made from thermoplastic materials, including polyethylene and polypropylene, and injected molded. The applicator members can be made from pulp or fibrous materials, with pulp injection molding techniques or spiral winding techniques, such as disclosed in U.S. Patent Application Nos. 63/701,862; 63/701,863; 63/701,866; and 63/701,867, which are all incorporated by reference herein. Some cardboard or cellulosic applicator members are made as a laminate with one or more inner layers made from paper stock or other fibrous material, and an outer layer being a film or a coating to reduce surface friction. In some embodiments, the different applicator members are made out of different materials and/or by different manufacturing methods. The different applicator members may be a single color or be different colors.

An exemplary tampon 20 is shown in FIG. 2. Tampon 20 comprises a primary absorbent 22, a secondary absorbent 30, and a withdrawal member 35. The primary absorbent 22 has a forward end 23 and a rearward end 24, which is opposite the forward end 23. The withdrawal member 35 may be joined to the primary absorbent member. The secondary absorbent 30 may be joined to at least one of the primary absorbent 22 and the withdrawal member 35.

The secondary absorbent 30 comprises a leading portion positioned between the forward end 23 and the rearward end 24 of the primary absorbent 22 or adjacent to the rearward end 24 of the primary absorbent 22. The secondary absorbent 30 comprises a trialing portion that is opposite the leading portion and extends rearward from a location proximate the rearward end 24 of the primary absorbent 22. Stated another way, the trialing portion of the secondary absorbent is that portion of the secondary absorbent beginning external to the rearward end of the primary absorbent to the end of the secondary absorbent, or that portion of the secondary absorbent that is external to the primary absorbent. The secondary absorbent may be structured such that the material is tapered. For example, the trailing portion may be tapered such that more material, a greater mass of material, is present in the area adjacent the rearward end 24 of the primary absorbent and the mass of material may be reduced as the trailing portion extends away from the rearward end 24. The withdrawal member 35 is configured to extend beyond the trailing portion of the secondary absorbent 30 so that the user is able to easily access the tampon product for removal. Tampons contemplated herein, however, are not limited to structures having the particular configuration shown in the figures.

Absorbent material utilized in primary absorbent 22 can include a wide variety of liquid-absorbing materials commonly used for absorbency in absorbent products, such as rayon fibers, cotton fibers, and/or comminuted wood pulp fibers (sometimes called “airfelt”). Examples of other suitable absorbent materials may include creped cellulose wadding; spun and/or meltblown polymer fibers or filaments; chemically stiffened, modified or cross-linked cellulosic fibers; other synthetic fibers such as polyamide fibers (e.g., nylon fibers); peat moss; absorbent foams (such as open-celled foam formed through polymerization of a high internal phase water-in-oil emulsion); nonwoven web materials of natural and/or synthetic fibers or combinations thereof, tissue including tissue wraps and tissue laminates; or any equivalent material or combinations of materials, or blends or combinations of these. Suitable rayon fibers may include but are not limited to viscose, MODAL, TENCEL (or lyocell). Rayon fibers may be tri-lobal and conventional rayon fibers. Rayon fibers may at least one of a round, trilobal, ellipsoid, stadium, and hollow cross sectional shape. It is to be appreciated that round fibers include those fibers with a perfectly circular cross sectional area and fibers that are substantially round with some minor deviation in the absolute radius as the fiber is assessed about the radius across the 360 degrees of the cross sectional shape. Rayon fibers may be needle punched or spunlace to form the primary absorbent. Suitable cotton fibers may include long fiber cotton, short fiber cotton, cotton linters, T-fiber cotton, card strips, and comber cotton. Preferably, the cotton fibers or fabric layer thereof should be scoured (for removal of natural hydrophobic waxes and impurities) and bleached (for whiteness) and may be imparted with a glycerin finish (for enhancing compaction), a leomin finish (for lubricity), or other suitable finish. In some embodiments, it may be desired that rayon or cotton or a blend thereof, forms the greater proportion (by weight) of the absorbent material, or that rayon alone forms the greater proportion (by weight) of the absorbent material, since rayon fibers may possess absorbency properties or capacity greater than those of other fibrous materials, per unit weight and/or per unit cost. For example, the primary absorbent may comprise greater than 50% by weight rayon fibers and less than about 50% by weight cotton fibers. More preferably, the primary absorbent may comprise greater than 80% by weight rayon fibers and less than about 20% by weight cotton fibers. The primary absorbent may comprise about 95% by weight rayon fibers and about 5% by weight of cotton fibers. The primary absorbent may comprise about 90% by weight rayon fibers and about 10% by weight of cotton fibers. The primary absorbent may comprise more than 95% by weight rayon and less than 5% by weight of cotton fibers. Additionally, superabsorbent materials, such as superabsorbent polymers or absorbent gelling materials may be incorporated into the primary absorbent.

The absorbent material of the primary absorbent 22 may be surrounded with a liquid permeable overwrap (not shown). Overwrap materials and processes may include rayon, cotton, spunlace, wetlaid, needlepunched, hydroentangled, adhesive bonded, thermal bonded, pressure bonded, spunbond monocomponent, bicomponent or multicomponent fibers, or other suitable natural or synthetic fibers known in the art. In some forms, a primary absorbent overwrap may be formed of a nonwoven web of spunbond fibers. The spunbond fibers may be spun from, for example, polymer resin including polyolefins such as polypropylene, polyethylene, or a blend or combination thereof. When formed of ordinarily hydrophobic materials such as polyolefins (including polypropylene and polyethylene) an overwrap material may be treated, e.g., by application of a suitable surfactant, to render it hydrophilic, so that it will readily attract and permit aqueous fluid to wick therethrough to the absorbent material within the wrapper. A nonwoven web material formed of polymeric material as described may be desired to form the overwrap, over natural fibrous materials or semi-synthetic rayon, for reasons of having a soft, smooth and comfortable feel and low friction against sensitive skin and internal tissues, relatively low cost, and superior wet structural integrity.

The primary absorbent 22 may have any suitable size, shape, and thickness that will both provide a suitable quantity of absorbent material and resulting absorption capacity, while permitting compression into a self-sustaining form of a size and shape suitable for easy and comfortable insertion. An uncompressed, opened size similar to those of conventional currently available tampons has been found to work well. A typical size for an uncompressed primary absorbent may be from about 2 cm to about 8 cm in longitudinal length and from about 3 cm to about 8 cm in lateral width, including any combination of length and width within those ranges, in combination with an uncompressed thickness anywhere from about 1 cm to about 3 cm. Total basis weight for a flat, uncompressed and open primary absorbent, may be from about 150 g/m² to about 1,400 g/m², calculated as the weight of the primary absorbent divided by the largest surface area on one side of the primary absorbent.

The primary absorbent of the present invention may be compressed into a generally cylindrical, self-sustaining form in the width direction, the radial direction, the axial direction, or any combination of these directions. Once compressed (as shown in FIG. 2), the primary absorbent may be inserted into an applicator. The compressed primary absorbent may have a compressed tampon length measured parallel to and along the longitudinal axis from the forward end to the rearward end of from about 30 mm to about 49 mm or from about 31 mm to about 51 mm. More specifically, a light absorbency, or light tampon, may have a compressed tampon length of from about 30 mm to about 38 mm. A medium absorbency, or regular tampon, may have a compressed tampon length of from about 39 mm to about 44 mm. A high absorbency, or super tampon, may have a compressed tampon length of from about 45 mm to about 49 mm. Once inserted into the vagina, the absorbent tampon 10 can expands upon fluid contact.

While the primary absorbent 22 may be formed into a substantially cylindrical configuration, such as illustrated in FIG. 2, other shapes are also possible. These may include shapes having a cross section which may be described as oval, elliptical, ovoid, stadium, rectangular, triangular, trapezoidal, semi-circular, or other suitable shapes. Non-limiting examples of suitable primary absorbent form, material composition, and structure are depicted and described in U.S. Patent Application Publication Nos. 2010/0268182 and US 2007/0260211.

The secondary absorbent 30 may be formed of fibrous material(s) similar to those used to form the primary absorbent. The secondary absorbent may be formed from polyester fibers, polyethylene fibers, polypropylene fibers, cotton fibers, rayon fibers, including viscose fibers and lyocell fibers, cotton fibers, or any combination thereof. In such examples the fibrous material forming the secondary absorbent should be configured such that the body of the secondary absorbent has less wicking potential than the primary absorbent. The wicking potential of the secondary absorbent can be adjusted by the selection of material of which it is formed, for its relative level of fiber surface hydrophilicity and its relative capillarity. Capillarity may be adjusted by the manner in which the fibers forming the secondary absorbent are consolidated and densified within in the structure.

It may be desired that the component fibrous material of the secondary absorbent 30 have a combination of fibers with hydrophilic surface properties, and optimized capillarity, to promote wicking of fluid therealong, but at the same time, not have, or not be assembled in a configuration having, a combination of capillarity and hydrophilicity that render it more likely to attract and retain menstrual fluid against the wicking potential of the material(s) of the primary absorbent 22. It is preferred that the material of the secondary absorbent serve to wick fluid to the primary absorbent, but that the material(s) of the primary absorbent have greater wicking potential and absorbency so as to be effective at drawing menstrual fluid from the secondary absorbent over the expected normal duration of use of the tampon.

Secondary absorbent 30 may be joined to at least one of the withdrawal member 35 and the primary absorbent 22. This joining of the secondary absorbent may occur subsequently to compression of the primary absorbent 22 to a self-sustaining form. In some variations it may be desirable to join some or all of the secondary absorbent 30 to the primary absorbent 22, the withdrawal member 35, or both, prior to compression of the primary absorbent 22 to a self-sustaining form.

The tampon withdrawal member 35 may be a separate section of cord, string, yarn, ribbon, knitted cord, or strip of woven or nonwoven fabric formed separately of the components of the primary absorbent and secondary absorbent, and then joined by any suitable mechanism to the primary absorbent and/or to the secondary absorbent. The joining may include sewing, adhesive attachment, thermal or pressure bonding, through-punching, penetration and/or looping of the withdrawal member about structure(s) of the primary absorbent or portions thereof, or any combination of these. A leading portion of the withdrawal member 35 may be joined to any suitable location on the primary absorbent 22, although it may be preferable that the joining location be substantially laterally centered on the primary absorbent and proximate to, or include a location proximate to, the rearward end 24 of the primary absorbent, so that tensile withdrawal force in the withdrawal member, exerted by the user, acts predominately on the rearward end of the primary absorbent and does not tend to substantially rotate or reorient the primary absorbent within the user's body during withdrawal.

The withdrawal member 35 may be formed of a strand or strands (such as two or more strands) of component yarn or thread material. In some examples the yarn or thread material may be formed of cotton fiber, cotton fiber processed or treated to be suitably hydrophobic, other natural plant-based fiber which may be processed or treated to be suitably hydrophobic, polyester, or a combination or blend thereof. The component yarn or thread may be knitted, twisted or braided to form the withdrawal member stock. For maximized tensile strength per unit decitex of the withdrawal member, it may be desired that the component yarn or thread be of twisted or braided construction (rather than of knitted, woven, or other construction).

Exemplary tampons comprising a secondary absorbent are disclosed in U.S. Pat. Nos. 6,258,075 and 6,599,279, which are herein incorporated by reference, and commercially available under the trade name of TAMPAX PEARL™. While FIG. 2 shows and the discussion above focuses on tampons embodying a secondary absorbent, other tampons configurations are contemplated herein that do not have a secondary absorbent component.

Tampons are typically designed in different sizes or absorbency levels to meet varying needs among different users and/or varying needs of a single user on different days of her period. “Syngyna” is an industry standard tampon absorbency capacity test. The apparatus and method for performing this tampon absorbency test are provided in 21 United States Code of Federal Regulation 801.430. In the United States and other regions, tampons carry standardized absorbency labels according to syngyna test absorbency ranges. Tampons absorbing less than 6 grams as “light absorbency,” tampons absorbing from 6 grams to 9 grams are labeled as “regular absorbency,” tampons absorbing 9 grams to 12 grams are labeled as “super absorbency,” tampons absorbing 12 grams to 15 grams are labeled as “super plus absorbency,” and tampons absorbing greater than 15 grams are labeled as “ultra absorbency.”

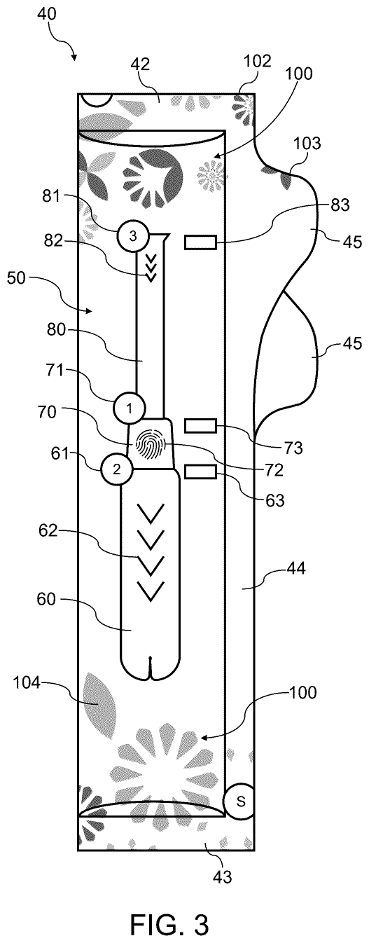

Referring now to FIG. 3, a primary package 40 in the form of a wrapper is illustrated. A wrapper, such as that shown in FIG. 3, can be made from a single sheet of material wherein the material is folded over to cover and seal a disposable absorbent product. The single sheet of material may be sealed on three edges 42, 43, and 44 to form an enclosure for the disposable absorbent product. Alternatively, a wrapper can be made from two or more sheets of material wherein individual material edges can be affixed to one another. The method of sealing edges is not critical, with exemplary modes including heat sealing, heat and pressure sealing, adhesive sealing, ultra sonic sealing, and the like.

Suitable wrappers can be constructed from numerous materials, including, for example, polymeric films, fibrous materials (including nonwovens and wovens), paper, card stock, and combinations thereof. In one form, the wrappers are constructed from flexible, polymeric films. The polymeric films may be based on polyethylene, polypropylene, polyester, nylon, polyvinyl alcohol, or blends of the same.

Primary packages according to the present invention can be formed of any suitable substrate material, such as, for example, a polymeric film comprising polyolefins, polyesters, polyamides, polyvinyl chlorides, ethylene-vinyl acetate copolymers, and/or other suitable films, a nonwoven, a formed film, a paper, or a fabric comprised of suitable mate rial such as polyethylene, polypropylene, polyester, cellulose, rayon, cotton, super absorbent material such as poly acrylate, or combinations thereof. The primary package substrate material can be any suitable thickness, such as, for example, greater than about 0.1 mm thick, such as, e.g., greater than about 0.2 mm thick, greater than about 0.3 mm thick, greater than about 0.4 mm thick, greater than about 0.5 mm thick, greater than about 0.6 mm thick, greater than about 0.7 mm thick, greater than about 0.8 mm thick, greater than about 0.8 mm thick, greater than about 0.9 mm thick, greater than about 1 mm thick, greater than about 2 mm thick, greater than about 3 mm thick, greater than about 4 mm thick, greater than about 5 mm thick, or any other suitable thickness.

The primary package substrate may comprise a single layer or component. In certain embodiments, the primary package substrate material can be a multiple component material that can have a first component and a second component. For example, the first component of the package material can be a polymeric film and the second component can be an absorbent material, such as, for example, a nonwoven, a formed film, a paper, or a fabric. The first component and the second component can be joined in any suitable manner to form the package substrate material, such as, for example, by adhesive bonding and/or mechanical bonding. In some forms, the substrate materials used in the primary package can have a basis weight of less than about 50 g/m², such as, for example, less than about 40 g/m², less than about 30 g/m², less than 20 g/m², or less than 10 g/m².

Various opening mechanisms can be employed, such as pull tabs 45 as shown in FIG. 3. In addition or alternative to pull tables 45, a line of weakness can be imparted into the wrapper material(s). The line of weakness can take many forms, including, but not limited to, a perforated line, a scored line, and an embossed line.

As discussed in the Background section above, primary packages for feminine hygiene products are traditionally designed with discretion in mind. For example, the primary packages can be configured so that one cannot see or ascertain the disposable absorbent product enclosed within the primary packages. Opaque materials can be employed to block a person' ability to see inside the primary packages, and/or decorative elements can be used on the primary packages to disguise the contents, for example. However, if an individual product and its primary package are isolated from the secondary package and information contained therein/thereon (for example, when the product is carried outside of the home), then a new user of the disposable absorbent product may be missing important or reassuring information about the product.

Applicator tampons can be specifically difficult, cumbersome, or intimidating to use by new users. An applicator needs to be gripped in a particular location. If a user grips an applicator too high, then the applicator and subsequently expelled tampon may not be ideally positioned within the vaginal canal, leading to the potential of being uncomfortable, ineffective, or prone to slipping out. Besides just resistance to further insertion by gripping an applicator too high, new users may not understand or routinely insert an applicator to an ideal depth due to lack of experience, sightline or other. This can result in poor tampon placement and render the tampon less effective than it otherwise could be. A user can also inadvertently grip an applicator too low whereby they clamp down on the tampon expulsion member such that it cannot slidably move in order to expel the tampon, potentially leading to frustration and waste.

To address the above and other issues and concerns, and with reference again to FIG. 3, primary packages 40 can include both a product zone 50 in addition to a design zone 100 on its outer surface(s). Product zone can include indicium, one or more images, or a combination thereof. Product zone 50 can include product zone elements that convey information related to a product feature, attribute, and/or operational aspect of a disposable absorbent product other than its size or absorbency level. The product zone can enable one to see or ascertain what is contained in the primary package without opening the package. Thus, the product zone may include transparent sections, windows, and the like in primary package materials. The product zone may also include visuals associated with an outer surface of the primary package.

Product zone 50 shown in FIG. 3 includes an image of an applicator comprising an applicator insertion member image 60, an applicator gripping member image 70, and a tampon expulsion member image 80. The size image of the applicator may be at least 50% of the size of the tampon applicator, such as illustrated in FIG. 1. More specifically, for any dimension taken on the image of the applicator disposed in the product zone, that same dimension, where applicable, is at least 50% of the dimension on the tampon applicator disposed within the primary package.

For each of the applicator member images, three different indicia are shown in FIG. 3. Applicator insertion member image 60 includes indicium 61 in the form of a number to convey an operational order aspect for example, indicium 62 in the form of a symbol or geometrical shape to help understand a proper applicator insertion end and insertion direction, and indicium 63 in the form of color or text (for example, “insert to here”), or other. Applicator gripping member image 70 includes indicium 71 in the form of a number related to operational aspects, indicium 72 in the form of non-textual indicium (for example, a fingerprint visual), and indicium 73 that can include textual indicium (for example, “hold”). Lastly, tampon expulsion member image 80 comprises indicium 81 in the form of a number, indicum 82 in the form of a symbol or geometrical shape to help understand pushing direction to expel a tampon, and indicium 83 in form of color or text (for example, “push”), or other. It should be appreciated that different indicia than that shown and described can be used in product zone 50, and also appreciated each applicator member image can include a single indicium, or any combination of the indicia shown and described. The indicium may be disposed on the applicator insertion member image 60, such as indicium 62, 72, and 82, and/or the indicium may be disposed adjacent to the applicator insertion member 60, such as indicium 63, 73, and 83. It is also be appreciated that indicum 63, 73, and 83 may be disposed on the applicator insertion member image 60.

Elements of product zone 50 may be spaced apart from, adjacent to, or overlap with elements of design zone 100. If overlapping, elements of the design zone should preferably not interfere with the communicative effectiveness of the product zone.

In some forms, at least part of design zone 100 can extend to and/or intersect with one or more sealed edges 42, 43, 44 of primary package 40. By way of example only, decorative elements 102, 103 exist within, and can even be truncated by, a sealed edge, while decorative element 104 is positioned away from a sealed edge. Product zone 50 is preferably, although not necessarily, spaced apart from the sealed edges, preferably by at least 2 mm, 3 mm, 4 mm, 5 mm, or more from the sealed edges. This can be accomplished through a variety of means, including, for example, online printing of the primary package wherein placement of a disposable absorbent product in relation to the primary package material is controlled, or through preprinting the primary package material and using registration marks or other visual control means for adjusting positioning of sections of primary package material as it is processed on a machine making packaged disposable absorbent products.

The product zone is preferably positioned at substantially the same location of the primary package among a plurality of primary packages contained within a single secondary package. The same location meaning a distance from a edge of the primary package, wherein the distance does not vary more than 1%, 2%, 5%, 10%, or 20% among ten primary packages within a single secondary package. In an alternative embodiment, the product zone location among ten primary packages can vary by more than 5%, 10%, 20%, 25%, or 30%. Where a feature of a disposable absorbent product (for example, an image of one or more members of a tampon applicator) is provided in the product zone, it is preferred there is only one of the feature; that is, the feature does not repeat. Alternatively, a product feature may repeat on an outer surface of the primary package.

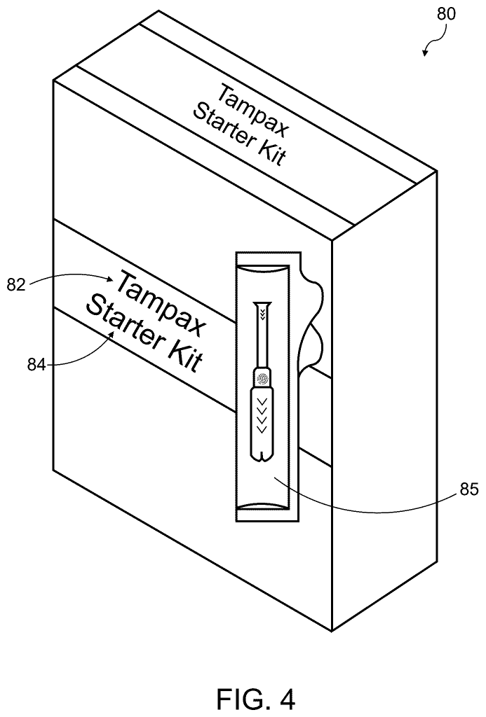

FIG. 4 illustrates an exemplary secondary package 80 in the form of a carton. While the carton is shown as generally rectangular in FIG. 4, other configurations are contemplated herein. And other secondary package forms such as, for example, bags and cannisters can be used. Suitable secondary packages can be made from paperboard, cardboard, plastic films, laminates, and the like. In some forms, the secondary package includes a reclosable portion and/or a lid or cover. Carton 80 is shown with a brand name 82, a communicative indicium 84, and a visual of a primary package similar to the one shown in FIG. 3. Communicative indicium 84 includes the text “Starter Kit” to alert potential purchasers that the package of disposable absorbent products contains additional assistance to help a new user have a positive and successful experience with the products. Other marketing language and visuals can also be employed with the secondary packages of the present invention to distinguish the packages from others offered for sale.

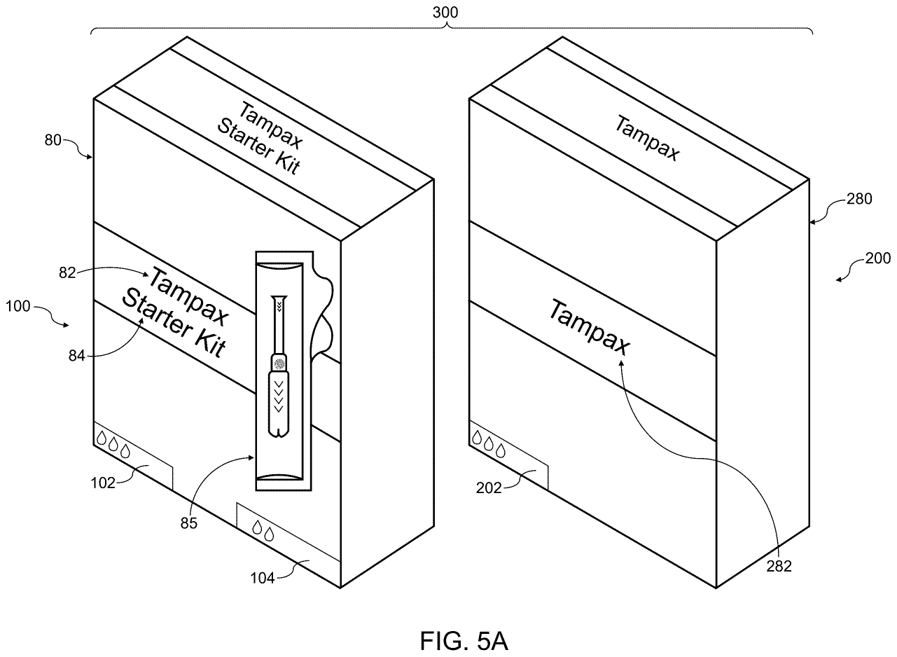

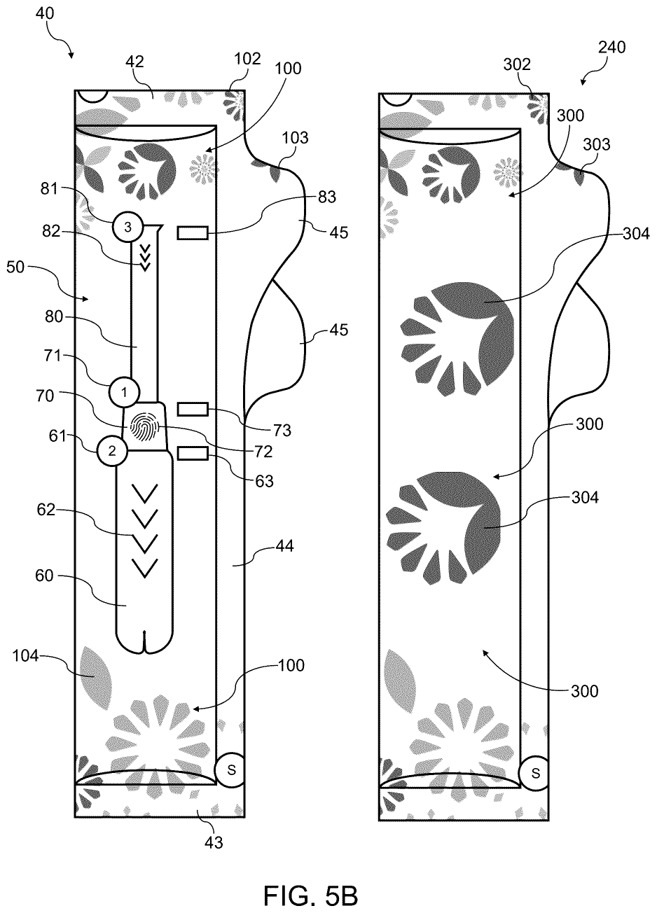

The present disclosure provides arrays of packaged disposable absorbent products. FIGS. 5A and 5B show one example of an array provided herein that includes a first package 100 of disposable absorbent products and a second package 200 of disposable absorbent products. First package carton 80 contains features like that in FIG. 4, which are labeled correspondingly. The first package carton 80 also includes indicium 102 related to a first size or absorbency level of a first disposable absorbent product contained in carton 80, and indicium 104 related to a second size or absorbency level of a second disposable absorbent product contained in carton 80. Indica 102 and 104 can include a color, a character (letter or number), a symbol(s), and other to represent size and/or absorbency level. It should be understood that the carton may only contain products of a single size or absorbency level, or may contain products having more than two sizes or absorbency levels.

Second package 200 includes a carton 280 with brand name 282 that is the same as brand name 82 associated with first package carton 80. Carton 280 includes indicium 202 related to a size or absorbency level of a disposable absorbent product contained therein. The disposable absorbent product of second package 200 is the same as one of the disposable absorbent products in first package 100. It should be understood that manufacturing variability will exist between individual products manufactured and sold. Being the “same” product means made with similar specifications, with similar materials, and/or with similar manufacturing approaches to produce products marketed and sold as the same product (carrying a same product name or brand name) to consumers. Variation can occur due to manufacturing conditions, equipment, raw material supply, machine settings, etc. For additional clarity, same products generally comprise attributes or properties within 10%, 5%, 1%, or less than 1% when individual products are measured or assessed according to an attribute or property. In one form, a first array package comprises a first tampon and a first tampon applicator, a second array package comprises a second tampon and a second tampon applicator, wherein at least one of the first tampon and the second tampon are the same, and the first tampon applicator and the second tampon applicator are the same.

FIG. 5B shows primary packages 40 and 240 associated with first package 100 and second package 200, respectively. Primary package 40 in the form of a wrapper is labeled with reference numerals corresponding to the wrapper shown in FIG. 3. Primary package 240, also in the form of a wrapper, comprises a design zone 300 that contains a plurality of decorative elements 302 and 303, for example. A product zone however is missing on wrapper 240, and instead, additional decorative elements 304 are exhibited on its outer surface. In this manner, design zone 300 can essentially exist over all or a majority of the primary package to provide discretion to a user carrying the product outside of the home. That is, one cannot see or ascertain what disposable absorbent product is contained in wrapper 240. Primary package 40 accordingly comprises an education format while primary package 240 comprises a discretion format.

A new user can purchase first package 100 to build confidence from the successful use of the disposable products contained therein. The new user can subsequently purchase second package 200 thereafter to gain a discretion benefit associated with the continued use of the same disposable absorbent product.

Methods of merchandising disposable absorbent products are also provided herein. An exemplary method comprises the steps of (a) providing a first package of disposable absorbent products, the first package comprising first primary packages each of which containing a first applicator and a first tampon disposed within the first applicator, and a first secondary package comprising a plurality of individual first primary packages; (b) providing a second package of disposable absorbent products, the second package comprising second primary packages each of which containing a second applicator and a second tampon disposed within the second applicator, and a second secondary package comprising a plurality of individual second primary packages; and (c) carrying out at least one of (i) offering for sale at the same time the first package of disposable absorbent products and the second package of disposable absorbent products; and (ii) shelving at the same time the first package of disposable absorbent products and the second package of disposable absorbent products. The first tampon is the same as the second tampon. The first primary packages are configured so that one can see the first applicator contained therein without opening the first primary packages. And the second primary packages are configured so that one cannot see the second applicator contained therein without opening the second primary packages. Preferably, the first secondary package and the second secondary package are marketed with a common product name or brand name. In one scenario, steps (a), (b), and (c)(ii) are completed by a retailer. Instead of shelving both the first package and the second package at the same time, a retailer can shelve the first package and offer for sale the second package on its website. In another scenario, steps (a), (b), and (c)(i) are completed by a manufacturer.

Arrays of packaged disposable absorbent products and methods of merchandising the same can involve shelving a first package of disposable absorbent products within close proximity to the second package of disposable absorbent products; for example, within 2, 3, 5, 10, 15 feet of one another. The first package can be placed adjacent to (beside, below, or above, for example) the second package.

While the figures and written description focus mainly on tampons, and specifically applicator tampons, other types of disposable absorbent products are contemplated herein including, but not limited to, sanitary napkins, maxi-pads, ultrathin pads, pantiliners, interlabial products, diapers, baby/toddler pants, and adult incontinence products such as pads, briefs, and pants.

### Exemplary Packages of Disposable Absorbent Products

A. A package of disposable absorbent products, the package comprising:
(a) a primary package comprising an applicator tampon;
(b) a secondary package comprising a plurality of the primary packages;
(c) a design zone on an outer surface of each of the primary packages, the design zone comprising a decorative element; and
(d) a product zone on the outer surface of each of the primary packages, the product zone comprising a product element.

B. A package according to Paragraph A, wherein the product zone is positioned in substantially the same location among the plurality of primary packages.

C. A package according to Paragraph A, wherein the product zone is positioned in a location that varies among the plurality of primary packages.

D. A package of applicator tampons, the package comprising:
(a) a primary package comprising an applicator tampon;
(b) a secondary package comprising a plurality of the primary packages; and
(c) an image of the applicator tampon disposed on an outer surface of the primary packages.

E. A package of applicator tampons, the package comprising:
(a) a primary package comprising an applicator tampon;
(b) a secondary package comprising a plurality of the primary packages; and
(c) features of the applicator provided on an outer surface of the primary packages.

F. A package of applicator tampons, the package comprising:
(a) a primary package comprising an applicator tampon;
(b) a secondary package comprising a plurality of the primary packages;
(c) an image of the applicator exhibited on an outer surface of the primary packages which comprises an applicator insertion portion, an applicator gripping portion, and a tampon expulsion portion; and
(d) indicium exhibited on the outer surface of the primary packages that is associated with at least one of the applicator insertion portion, the applicator gripping portion, and the tampon expulsion portion;
(e) wherein the indicium comprises alphanumeric characters.

G. A package of applicator tampons, the package comprising:
(a) a primary package comprising an applicator tampon;
(b) a secondary package comprising a plurality of the primary packages;
(c) an image of the applicator shown on an outer surface of the primary packages which comprises an applicator insertion portion, an applicator gripping portion, and a tampon expulsion portion; and
(d) an image of the applicator also shown on an outer surface of the secondary package.

H. A package of applicator tampons, the package comprising:
(a) a primary package comprising an applicator tampon, wherein the applicator comprises an applicator insertion portion, an applicator gripping portion, and a tampon expulsion portion;
(b) a secondary package comprising a plurality of the primary packages;
(c) a leaflet disposed within the secondary package which comprises information related to the applicator insertion portion aspects, the applicator gripping portion aspects, and the tampon expulsion portion aspects to inform a user of the applicator tampon when the secondary package is opened; and
(d) at least some of the plurality of aspects provided on an outer surface of the primary package to inform the user of the applicator tampon when an individual primary package is removed from the secondary package.

I. A package of applicator tampons, the package comprising:
(a) a primary package comprising an applicator tampon, wherein the applicator comprises an applicator insertion portion, an applicator gripping portion, and a tampon expulsion portion;
(b) a secondary package comprising a plurality of the primary packages;
(c) an outer surface of each of the primary packages comprising a design zone and a product zone;
(d) wherein the design zone comprises a decorative element;
(e) wherein the product zone comprises at least one of the applicator insertion portion, the applicator gripping portion, and the tampon expulsion portion to inform a user of the applicator features without having to open the primary packages.

J. A package of disposable absorbent products, the package comprising:
(a) a primary package comprising a disposable absorbent product;
(b) a secondary package comprising a plurality of the primary packages; and
(c) at least 10% of an outer surface area of the primary package contains a disposable absorbent product attribute other than size or absorbency.

K. A package of disposable absorbent products, the package comprising:
(a) a primary package comprising a tampon and an applicator for positioning the tampon within a body, the primary package comprising a package length;
(b) a secondary package comprising a plurality of the primary packages; and
(c) a product zone on the outer surface of each of the primary packages, the product zone comprising an applicator image;
(d) wherein the applicator comprises a length that is a first percent length of the package length;
(e) wherein the applicator image comprises a length that is a second percent length of the package length; and
(f) wherein the first percent length is greater than the second percent length.

L. A package of disposable absorbent products, the package comprising:
(a) a primary package comprising a disposable absorbent product;
(b) a secondary package comprising a plurality of the primary packages; and
(c) an image of the disposable absorbent product disposed on an outer surface of the primary package so that it is clear what is contained within the primary package.

M. A package of disposable absorbent products, the package comprising:
(a) a primary package comprising a disposable absorbent product; and
(b) a secondary package comprising a plurality of the primary packages;
(c) wherein each of the primary packages are configured so that a user can see what is contained within the primary packages without opening them.

N. A package according to any one of the preceding paragraphs, wherein the primary package comprises post-consumer recycled content.

O. A package according to any one of the preceding paragraphs, wherein the secondary package comprises post-consumer recycled content.

P. A package according to any one of the preceding paragraphs, wherein the primary package comprises a bio-based polymer.

### Exemplary Arrays of Packaged Disposable Absorbent Products

A. An array of packaged applicator tampons, the array comprising:
(a) a first package comprising a first applicator and a first tampon disposed within the first applicator; and
(b) a second package comprising a second applicator and a second tampon disposed within the second applicator;
(c) wherein at least one of:
   (i) the first applicator is the same as the second applicator; and
(ii) the first tampon is the same as the second tampon;
(d) wherein the first package comprises an education format;
(e) wherein the second package comprises a discretion format;
(f) wherein the first applicator, the first tampon, the second applicator, and the second tampon are manufactured for or by a single company.

B. The array of Paragraph A, wherein the first package is marketed to a new user of the first applicator and the first tampon.

C. The array of Paragraph A, wherein the first package is contained within a first secondary package, wherein the second package is contained within a second secondary package, and wherein the first secondary package and the second secondary package share a common product name and/or brand name.

D. The array of Paragraph A, wherein the first package and the second package are offered for sale at the same time.

E. The array of Paragraph A, wherein the first package and the second package are positioned on a store shelf at the same time.

F. The array of Paragraph A, wherein the first package and the second package are offered for sale on a single online website.

G. An array of packaged applicator tampons, the array comprising:
(a) a first package of applicator tampons, the first package comprising first primary packages each of which containing a first applicator tampon, and a first secondary package comprising a plurality of the first primary packages; and
(b) a second package of applicator tampons, the second package comprising a second primary package each of which containing a second applicator tampon, and a second secondary package comprising a plurality of the second primary packages;
(c) wherein an outer surface of each of the first primary packages comprises:
   (i) an applicator image comprising an applicator insertion member, an applicator gripping member, and a tampon expulsion member; and
   (ii) indicium located on at least one of an area proximate the applicator insertion member, an area proximate the applicator gripping member, and an area proximate the tampon expulsion member;
(d) wherein an outer surface of each of the second primary packages is devoid of both the applicator image and the indicium associated with the first primary packages;
(e) wherein the first applicator tampon is the same as the second applicator tampon; and;
(f) wherein the first secondary package and the second secondary package comprise the same product name or brand name.

H. An array of packaged disposable absorbent products, the array comprising:
(a) a first package of disposable absorbent products, the first package comprising first primary packages each of which containing a first disposable absorbent product, second primary packages each of which containing a second disposable absorbent product that is a different size or absorbency level than that of the first disposable absorbent product, and a first secondary package comprising a plurality of the first primary packages and the second primary packages;
(b) a second package of disposable absorbent products, the second package comprising a third primary package each of which containing a third disposable absorbent product that is the same as either the first disposable absorbent product or the second disposable absorbent product, and a second secondary package comprising a plurality of the third primary packages;
(c) wherein an outer surface of each of the first primary packages and the second primary packages comprises:
   (i) a first location comprising decorative elements; and
   (ii) a second location comprising educational elements regarding features or aspects of the disposable absorbent product;
(d) wherein an outer surface of each of the third primary packages comprises:
   (i) the first location comprising decorative elements; ;
   (ii) the second location comprising additional decorative elements and/or being devoid of the educational elements associated with the first primary packages and the second primary packages;
(e) wherein the first secondary package and the second secondary package share a common product name or brand name.

I. An array of packaged applicator tampons, the array comprising:
(a) a first package of applicator tampons, the first package comprising first primary packages each of which containing a first applicator tampon, and a first secondary package comprising a plurality of the first primary packages; and
(b) a second package of applicator tampons, the second package comprising a second primary package each of which containing a second applicator tampon, and a second secondary package comprising a plurality of the second primary packages;
(c) wherein an outer surface of each of the first primary packages comprises:
   (i) a decorative zone;
   (ii) an educational zone;
(d) wherein an outer surface of each of the second primary packages comprises a decorative zone, but not an educational zone;
(e) wherein the first applicator tampon is the same as the second applicator tampon; and;
(f) wherein the first secondary package and the second secondary package coproduct share a common product name or brand name.

J. An array of packaged disposable absorbent products, the array comprising:
(a) a first package comprising a first primary package, a first disposable absorbent product contained within the first primary package, and a first secondary package comprising a plurality of the first primary packages; and
(b) a second package comprising a second primary package, a second disposable absorbent product contained within the second primary package, and a second secondary package comprising a plurality of the second primary packages;
(c) wherein the first primary packages are configured so that a person can see or ascertain what is contained therein without opening the primary packages;
(d) wherein the second primary packages are configured so that a person cannot see or ascertain what is contained therein; and
(e) wherein the first disposable absorbent product is the same as the second disposable absorbent product.

K. An array of packaged disposable absorbent products, the array comprising:
(a) a first package comprising a first feminine care product, wherein the first package conveys application techniques;
(b) a second package comprising a second feminine care product, wherein the second package does not convey application techniques;
(c) wherein the first and second feminine care products have at least one of a size or absorbency level within 10% of each other;
(d) wherein the first and second packages comprise a common single source identifier; and
(e) wherein the first and second packages comprise different sub-brand name portions.

L. An array of packaged disposable absorbent products, the array comprising:
(a) a first package comprising a first feminine care product and a second feminine care product, wherein the first package conveys application techniques, wherein the first feminine care product is disposed within a first wrapper and the second feminine care product is disposed within a second wrapper, and wherein each of the first wrapper and the second wrapper conveys application techniques;
(b) a second package comprising a third feminine care product, wherein the third feminine care product is disposed within a third wrapper, and wherein the third wrapper does not convey application techniques;
(c) wherein the first and third feminine care products have at least one of a size or absorbency level within 10% of each other;
(d) wherein the second and third feminine care products have at least one of a size or absorbency level that is greater than 10% of each other; and
(e) wherein the first and second packages comprise a common single source identifier.

### Exemplary Methods of Merchandising Disposable Absorbent Products

A. A method of merchandising disposable absorbent products, the method comprising the steps of:
(a) offering for sale a first secondary package of applicator tampons comprising a first primary package comprising a first applicator and a first tampon disposed within the first applicator; and
(b) offering for sale a second secondary package of applicator tampons comprising a second package comprising a second applicator and a second tampon disposed within the second applicator; and
(c) wherein the first package secondary package is marketed to new users of the first applicator and the first tampon;
(d) wherein at least one of:
   (i) the first applicator is the same as the second applicator; and
   (ii) the first tampon is the same as the second tampon;
(e) wherein the first primary package comprises an education format;
(f) wherein the second primary package comprises a discretion format; and
(g) wherein the first secondary package and the second secondary package share a common product name and/or brand name.

B. The method of Paragraph, wherein steps (a) and (b) are conducted at the same time and/or the same location.

C. A method of merchandising disposable absorbent products, the method comprising the steps of:
(a) providing a first package of disposable absorbent products, the first package comprising first primary packages each of which containing a first disposable absorbent product, and a first secondary package comprising a plurality of the first primary packages;
(b) providing a second package of disposable absorbent products, the second package comprising second primary packages each of which containing a second disposable absorbent product, and a second secondary package comprising a plurality of the second primary packages;
(c) wherein the first disposable absorbent product is the same as the second disposable absorbent product;
(d) marketing the first package to a new user of the disposable absorbent product; and
(e) marketing the second package to the new user for a subsequent purchase of the disposable absorbent product;
(f) wherein the first secondary package and the second secondary package share a common product name or brand name.

D. A method of merchandising disposable absorbent products, the method comprising the steps of:
(a) providing a first package of disposable absorbent products, the first package comprising first primary packages each of which containing a first disposable absorbent product, and a first secondary package comprising a plurality of the first primary packages; and
(b) providing a second package of disposable absorbent products, the second package comprising second primary packages each of which containing a second disposable absorbent product, and a second secondary package comprising a plurality of the second primary packages;
(c) wherein the first disposable absorbent product is the same as the second disposable absorbent product;
(d) wherein the first primary packages are configured to educate a new user on aspects of the disposable absorbent product without having to open the first primary packages; and
(e) wherein the second primary packages are configured for discretion for what is contained therein for a subsequent use by the new user of the disposable absorbent product;
(f) wherein the first secondary package and the second secondary package are manufactured by and/or offered for sale by the same entity.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be understood by those skilled in the art that various other changes and modifications can be made without departing from the spirit and scope of the present disclosure. It is therefore intended to cover in the appended claims all such changes and modifications that are within the scope of the present disclosure.

What is claimed is:

1. A package of disposable absorbent products, the package comprising:
a) a primary package comprising an applicator tampon;
b) a secondary package comprising a plurality of the primary packages; and
c) a design zone on an outer surface of each of the primary packages, the design zone comprising a decorative element; and
d) a product zone on the outer surface of each of the primary packages, the product zone comprising a feature, attribute, and/or operational aspect of the applicator tampon other than size or absorbency level.

2. The package of claim 1, wherein the feature, attribute, and/or operational aspect of the applicator tampon is distal to the sealed edge.

3. The package of claim 1, wherein the feature, attribute, and/or operational aspect of the applicator tampon is spaced apart from the sealed edge by at least 3 millimeters.

4. The package of claim 1, wherein the decorative element is proximate to the sealed edge.

5. The package of claim 1, wherein the decorative element intersects the sealed edge.

6. The package of claim 1, wherein the feature, attribute, and/or operational aspect of the applicator tampon relates to at least one of an applicator insertion member, an applicator gripping member, and a tampon expulsion member.

7. The package of claim 6, wherein alphanumeric indicium is associated with each of the applicator insertion member, the applicator gripping member, and the tampon expulsion member.

8. The package of claim 1, wherein the feature, attribute, and/or operational aspect of the applicator tampon comprises ordered operational aspects.

9. The package of claim 1, wherein the secondary package also comprises the feature, attribute, and/or operational aspect of the applicator tampon.

10. The package of claim 1, wherein the package is marketed to a new use of the applicator tampon.

11. An array of packaged disposable absorbent products, the array comprising:
a) a first package comprising a first primary package containing a first applicator tampon, and a first secondary package comprising a plurality of the first primary packages; and
b) a second package comprising a second primary package containing a second applicator tampon, and a second secondary package comprising a plurality of the second primary packages;
c) wherein an outer surface of each of the first primary packages comprises:
i) a color and/or character related to an absorbency level or size of the first applicator tampon; and
ii) an aspect related to at least one of an applicator insertion member, an applicator gripping member, and a tampon expulsion member;
d) wherein an outer surface of each of the second primary packages comprises the same color and/or character associated with the first primary packages, but is devoid of the aspect related to at least one of an applicator insertion member, an applicator gripping member, and a tampon expulsion member associated with the first primary packages;
e) wherein the first applicator tampon is the same as the second applicator tampon; and;
f) wherein the first secondary package and the second secondary package share a common product name or brand name.

12. The array of claim 11, wherein the aspect related to at least one of an applicator insertion member, an applicator gripping member, and a tampon expulsion member comprises an image of the applicator.

13. The array of claim 12, wherein indicium comprising letters and/or numbers are associated with the applicator insertion member, the applicator gripping member, and the tampon expulsion member.

14. The array of claim 12, wherein an orientation of the image of the applicator is aligned with that of the applicator tampon within the primary packages.

15. The array of claim 11, wherein the aspect related to at least one of an applicator insertion member, an applicator gripping member, and a tampon expulsion member relates to a gripping location, an insertion depth, and tampon expulsion method.

16. A method of merchandising disposable absorbent products, the method comprising the steps of:
a) providing a first package of disposable absorbent products, the first package comprising first primary packages each of which containing a first applicator and a first tampon disposed within the first applicator, and a first secondary package comprising a plurality of the first primary packages;
b) providing a second package of disposable absorbent products, the second package comprising second primary packages each of which containing a second applicator and a second tampon disposed within the second applicator, and a second secondary package comprising a plurality of the second primary packages; and
c) carrying out at least one of:
i) offering for sale at the same time the first package of disposable absorbent products and the second package of disposable absorbent products; and
ii) shelving at the same time the first package of disposable absorbent products and the second package of disposable absorbent products;
d) wherein the first tampon is the same as the second a tampon;
e) wherein the first primary packages are configured so that a person can see the first applicator contained therein without opening the first primary packages; and
f) wherein the second primary packages are configured so that a person cannot see the second applicator contained therein without opening the second primary packages.

17. The method of claim 13, wherein steps (a), (b), and (c)(ii) are completed by a retailer.

18. The method of claim 13, wherein steps (a), (b), and (c)(i) are completed by a manufacturer.

19. The method of claim 13, wherein step (c)(i) is completed via an online website.

20. The method of claim 13, wherein the first secondary package and the second secondary package are marketed with a common product name or brand name.
